(19) United States
(12) Patent Application Publication
(10) Pub. No.: US 2006/0057187 A1
(43) Pub. Date: Mar. 16, 2006

(54) Emulsions containing unsaturated fatty acids and esters thereof

(76) Inventors: Rainer Eskuchen, Langenfeld (DE); Juergen Gierke, Illertissen/Betlinshausen (DE); Peter Horlacher, Bellenberg (DE)

Correspondence Address: COGNIS CORPORATION;PATENT DEPARTMENT, 300 BROOKSIDE AVENUE, AMBLER, PA, 19002, US

(21) Appl. No.: 11/215,709

(22) Filed: Aug. 30, 2005

(30) Foreign Application Priority Data
Sep. 10, 2004 (DE) 10 2004 043 824.2

(51) Int. Cl. A61K 47/00; A23L 2/38

(52) U.S. Cl. 424/439; 426/590

(57) ABSTRACT

The disclosed invention relates to emulsions containing, based on the total weight of the emulsion, a) 0.1 to 50% by weight of a conjugated linoleic acid or derivative thereof, b) 0.1 to 30% by weight of a vegetable lecithin mixture, c) 0 to 20% by weight of a co-emulsifier, d) 0 to 70% by weight of a polyol, e) 0 to 70% by weight of a carbohydrate, f) 0 to 1% by weight of an antioxidant, and g) 20 to 99% by weight of an aqueous phase, with the proviso that the emulsion contains no other oils and fats except for the triglycerides of components a), b) and/or c) optionally present. The emulsions may be directly processed in foods, more particularly beverages, and show high stability in storage.

### CROSS-REFERENCE TO RELATED APPLICATIONS

This application claims priority under 35 U.S.C. § 119 from German patent application no.10 2004 043 824.2, filed Sep. 10, 2004.

### BACKGROUND OF THE INVENTION

1. Field of the Invention

This invention relates generally to foods and, more particularly, to emulsions containing conjugated linoleic acid and derivatives thereof, to a process for the production of the emulsions and to foods, more particularly beverages, containing the emulsions.

2. Background Art

The use of conjugated linoleic acid (CLA) and its derivatives in foods and pharmaceutical preparations has been known for years. CLA and its derivatives have mainly been marketed as a food supplement in powder or capsule form. Increasingly, the dietetic is even being directly incorporated in foods. Thus, it is hoped to offer CLA and CLA compounds in beverages and dairy products and a search is being conducted for a processing form of the lipophilic compound which would be suitable for formulation in water-containing food bases.

In pharmaceutical preparations, emulsions and lotions often serve as vehicles for supplying the body with unsaturated fatty acids or derivatives. For example, emulsions containing CLA, amino acids and water are known from International patent application WO 2004/045506 A2. A CLA-containing emulsion for oral application, which additionally contains omega-3 fatty acids and vitamins, is described in U.S. patent application US 2004/0037892 A1. International patent application WO 02/070014 A1 discloses oil-in-water emulsions which contain conjugated linoleic acid, isomers or derivatives thereof and which may be used for parenteral, oral or topical application in human beings and animals. High-purity phospholipids are often used as emulsifiers for pharmaceutical applications, but are to be avoided for use in foods for reasons of cost. The use of animal-based emulsifiers in foods is often avoided on ethical grounds. Emulsions containing a particular antioxidation system consisting of lecithins and citrates, which stabilize oil-containing emulsions with high levels of unsaturated fatty acids, are known from the food sector, for example, as described in EP 0 884 952 B1. International patent application WO 00/21379, which also emanates from the food sector, claims oil-in-water emulsions based on dairy products which contain mono-, di- and triglycerides of CLA.

The problem addressed by the present invention was to provide CLA and CLA derivatives in a physicochemically stable formulation which would enable conjugated linoleic acid and its derivatives to be simply and inexpensively incorporated in beverages and dairy products. Further, the addition of the CLA formulation should not adversely affect the shelf life or the organoleptic properties of the CLA-enriched beverages and dairy products.

### BRIEF SUMMARY OF THE INVENTION

The present invention relates to emulsions containing
a) 0.1 to 50% by weight conjugated linoleic acid or derivatives of conjugated linoleic acid,
b) 0.1 to 30% by weight vegetable lecithin mixtures,
c) 0 to 20% by weight co-emulsifiers,
d) 0 to 70% by weight polyols,
e) 0 to 70% by weight carbohydrates,
f) 0 to 1 % by weight antioxidants, and
g) 20 to 99% by weight aqueous phase, with the proviso that the emulsion contains no other oils and fats except for the triglycerides of components a), b) and/or c) optionally present.

The emulsions with the above composition are intended to be incorporated in fruit juices, fruit juice mixtures, fruit juice beverages, vegetable juices, carbonated and non-carbonated beverages, soya milk beverages or protein-rich liquid food substitute beverages and dairy products, such as milk, fermented milk preparations, yogurt, drinking yogurt or cheese preparations.

### DETAILED DESCRIPTION OF THE INVENTION

Surprisingly, the emulsions have excellent storage stability and may readily be incorporated in the beverages and dairy products without any separation, i.e. creaming up, sedimentation or ring formation, of the CLA-containing products occurring. Since the more lipophilic part of the emulsion consists almost exclusively of CLA and/or its derivatives, the formulations lend themselves very well to incorporation in water-based beverages, in which they are present in finely dispersed form, without leading to unattractive and flavor-spoiling floating of the oil component after prolonged storage of the beverages. An additional advantage is the possibility of directly using the beverages as water phase in the production of the emulsions.

Accordingly, the present invention also relates to beverages and dairy products which contain emulsions with the composition shown above. The beverages or dairy products advantageously contain the beverages or dairy products in quantities of 0.1 to 20% by weight, preferably in quantities of 0.5 to 10% by weight and more particularly in quantities of 1 to 5% by weight, based on the weight of the CLA-containing food.

Production

The lecithin and optionally the co-emulsifiers are dissolved or pre-dispersed in the “aqueous” phase with stirring at 20 to 70° C. Other formulation ingredients, except for the conjugated fatty acid/fatty acid derivatives, are then dissolved or dispersed in the “aqueous” phase. The CLA or CLA derivatives is/are then added and optionally pre-homogenized using high-speed mixers or rotor/stator homogenizers, preferably an Ultra-Turrax. The emulsion may then be homogenized in another process step using another high-performance homogenizer. The emulsion may be subjected to this process step several times. The energy input is approximately 1×10⁵ to 2×10⁸ J m-3.

Suitable homogenizers are high-pressure dispersion systems such as, for example, radial diffusors with a flat or serrated valve; counter-jet dispersers such as the Microfluidizer for example; jet dispersers or orifice systems. Other suitable dispersion systems are rotor/stator systems, ultrasound systems, ball mills or membranes. Rotor/stator systems, for example colloid mills or gear ring dispersing machines, are preferably used for pre-dispersion.

The process also enables the emulsifiers and optionally co-emulsifiers containing the CLA and/or the CLA derivatives to be directly pre-emulsified in the beverages or thinly liquid dairy products as aqueous phase using the rotor/stator system and the high-pressure emulsification, for example, to be carried out in a following step. Depending on the concentration of the starting materials used, a CLA-containing beverage concentrate or even the final CLA-containing end product can thus be produced.

Besides the ingredients used, more particularly the emulsifiers, the dispersion process is crucially important to the physicochemical stability of the emulsions because it results in a particular particle size distribution of the emulsified droplets which in turn determines the short-term and long-term stability of the emulsions.

Particle Size Determination

Emulsions are thermodynamically unstable because they have a high interface and hence high interfacial energy, depending on the particle size of the inner phase. Nevertheless, they are regarded as stable if their degree of dispersity does not change significantly with the storage time or under stress conditions. In the case of orally applied emulsions used in the food sector, much larger particle sizes can be accepted than in the pharmaceutical sector of the parenteralia. Nevertheless, it has been found that emulsions with a relatively small initial particle size have far higher ultimate stability and that the time required to reach the maximum tolerable particle size and the time elapsing before creaming up and hence the storage time can be significantly extended. In addition, emulsions with a very small particle size distribution withstand stress tests, such as freezing/thawing cycles, autoclaving or mechanical loads, for example in an ultracentrifuge, far more successfully. Accordingly, it is essential for determining stability to measure the particle size distribution of emulsions immediately after their production and at certain time intervals.

The particle size distribution was measured with a Beckman Coulter LS 230 using the optical model emulsion.rfd PIDS inclded (of 14.08.01) in accordance with the operating instructions (1994). Water was used as the measuring medium. The particle size measurements were carried out immediately after production of the dispersions. Selected dispersions were subjected to a storage test (see Examples).

Conjugated Linoleic Acid and Derivatives of Conjugated Linoleic Acid

The term “conjugated linoleic acid” (CLA) encompasses all positional and structural isomers and mixtures of two or more isomers of linoleic acid or octadecadienoic acid containing two conjugating carbon double bonds, i.e. all cis and trans isomers (“E/Z isomers”) of 2,4-octadecadienoic acid, 4,6-octadecadienoic acid, 6,8-octadecadienoic acid, 7,9-octadecadienoic acid, 8,10-octadecadienoic acid, 9,11-octadecadienoic acid and 10,12 octadecadienoic acid, 11,13 octadecadienoic acid.

Above all, the C18:2 cis-9, trans-11 and C18:2 trans-10, cis-12 isomers, which are biologically the most active isomers, are of particular interest.

Pure CLA is often obtained by saponification of oils containing linoleic acid. In order to better control the content of isomers, the corresponding esters may also be used as starting products. The prior art in this regard is the production of the corresponding esters by esterification of the fatty acids with methanol or ethanol. According to the literature, linoleic acid methyl and ethyl esters in particular are suitable starting materials for mild conjugation, for example, WO 99/47135.

In the isomerization, the linoleic acid is rearranged mainly to the c9,t11 and t10,c12 isomer which, with a carefully controlled reaction, applies to up to 90% of the isomers. Advantageously, less than 1% of the isomers are then present as 11,13 isomers, less than 1% as 8,10 isomers and less than 1% as trans,trans isomers (t9,t11 and t10,t12 isomers) and less than 1% as unidentified compounds. A process such as described in German patent application DE 102 36086 results in a ratio of the main isomers cis-9,trans-11 and trans-10,cis-12 in approximately equal parts of 1:1.2 to 1.2:1.

Besides conjugated linoleic acid alcohol, the derivatives of conjugated linoleic acid preferably include the esters. Linoleic acid lower alkyl esters with an acyl group of conjugated linoleic acid and a linear or branched C₁₋₅ alkyl chain are known. Conjugated linoleic acid methyl and/or ethyl esters in particular are used. These esters may advantageously be produced by the process described in International patent application WO 03/022964.

Esters of glycerol with CLA, such as mono-, di- and in particular triglycerides of conjugated linoleic acid, are particularly preferred. The particularly preferred triglycerides are also known under the commercial name of Tonalin® TG 80 (Cognis Deutschland GmbH & Co. KG). CLA triglycerides can be produced by the processes described by applicants in German patent DE 197 18245 C2 and in International patent application WO 03/022964.

The conjugated linoleic acid and/or its derivatives are used in the emulsions in a quantity of 0.1 to 50% by weight, preferably in a quantity of 0.5 to 30% by weight, more preferably in a quantity of 1 to 25% by weight and most preferably in a quantity of 3 to 20% by weight, based on the total weight of the emulsion.

Vegetable Lecithin Mixtures

Chemically, the term lecithin is used for phosphatidyl choline (PC). Generally, however, the term lecithin is used for mixtures of substances which, besides the phospholipids (for example phosphoglycerides, sphingomyelins) as actual active principles, also contain, for example, oils, sterols or carbohydrates (see Ullmann'Encyclopedia of Industrial Chemistry, 2002, Author: Hiroyuki Tanno—Internet Online). Commercially available lecithins are marketed, for example, as purified, deoiled, hydrogenated, hydrolyzed or more or less highly enriched (with certain active principles)/purified products. Phospholipids are constituents of the cell membranes of all organisms and can be found in large quantities in egg yolk, the brain and vegetable seed cells. 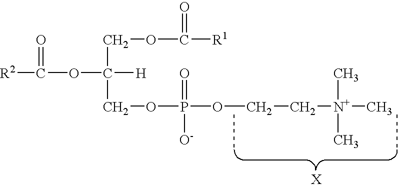
FIG. 1: structure of phospholipids; R¹, R² are unbranched aliphatic radicals containing 11 to 22 and preferably 15 to 18 carbon atoms and 0 to 4 cis-double bonds, X corresponds to the particular phosphatidyl component, here phosphatidyl choline. Ethanolamine, inositol and serine, for example, are typical.

The variety of fatty acid residues R¹ and R² results in a large number of different phospholipids. Extractions from biological material always yield mixtures which are further purified for use in pharmaceutical formulations. Thus, a fraction from soya beans also contains palmitic acid, stearic acid, palmitoleic acid, oleic acid, linoleic acid and linolenic acid. The composition of soya lecithin varies very widely. It consists, for example, of 15 to 50% phosphatidyl choline, 8 to 20% phosphatidyl ethanolamine, ca. 5 to 21% phosphatidyl inositol, 1 to 2% phosphatidyl serine, sterols, fatty acids, carbohydrates and fats. By contrast, lecithin obtained from egg yolk consists essentially of phosphatidyl choline. The lecithins obtained from soya beans, seeds and—for pharmaceutical preparations—mainly from egg yolk are used as emulsifiers primarily in the food industry, i.e. in margarine, chocolate, confectionery and coatings.

In contrast to the known CLA-containing emulsions, in which egg lecithins, phosphatidyl choline or phospholipids with a high phosphatidyl choline content and chemically highly pure isolated phospholipids are used (for example, WO 02/070014), only vegetable lecithin mixtures are used as lecithins in the present invention. These lecithin mixtures may be deoiled, hydrogenated, hydrolyzed or enriched with phosphatidyl choline or phosphatidyl inositol. These mixtures mainly emanate from soya oil, but not exclusively so far as the present invention is concerned. Surprisingly, more stable emulsions can be produced by using mixtures of different lecithins as opposed to highly pure lecithins. In addition, the use of lecithin mixtures provides for relatively inexpensive production for the food sector.

The lecithins preferably used have a phosphatidyl choline content (inc. lysophosphatidyl choline) of less than 80% and, more particularly, at most 75%. The vegetable lecithin mixtures are used in the emulsions in quantities of 0.1 to 30% by weight, preferably in quantities of 0.2 to 20% by weight and more particularly in quantities of 0.5 to 10% by weight.

Suitable commercial preparations are, for example,

| | |
|---|---|
| Premium ® IPM | new name Leciprime ® 1800 IP |
| Lipoid ® S 75 | ca. 75% phosphatidyl choline |
| Phospholipon ® 80 | ca. 76% phosphatidyl choline |
| Emultop ® HL 50 | deoiled, enzyme-hydrolyzed lecithin, ca. 14% |
| | phosphatidyl choline |
| Lipotin ® NE | hydrolyzed lecithin |

Co-Emulsifiers

Suitable co-emulsifiers are any toxicologically safe, food-grade emulsifiers which can be used in combination with the lecithin mixtures. Preferred co-emulsifiers are polyethylene glycol sorbitan fatty acid esters (Tweens), sorbitan fatty acid esters (Spans), glycerides, sucrose esters and poloxamers (Pluronics), among which polyethylene glycol sorbitan fatty acid esters, also known as polysorbates, and sucrose esters are particularly preferred. Polysorbates, such as Polysorbate 80 and Polysorbate 60 for example, are especially suitable.

An excessively high content of polysorbates, which gives the beverages a bitter taste, can be avoided by the use of vegetable lecithin mixtures, so that a combination of the lecithin mixtures with polysorbates has proved to be particularly effective.

Co-emulsifiers do not have to be present in the formulation. They are used in the emulsions in quantities of 0 to 20% by weight, based on the emulsion, preferably in quantities of 0.05 to 15% by weight and more particularly in quantities of 0.5 to 10% by weight.

Polyols

The polyols optionally used also have to be toxicologically safe and suitable for use in foods. Such polyols as, for example, glycerol, propylene glycol, sorbitol, D-mannitol or xylitol may be used, glycerol and sorbitol being particularly preferred polyols. Suitable quantities are 0 to 70% by weight, based on the total weight of the emulsion. Quantities of 10 to 70% by weight are preferred and quantities of 30 to 60% by weight particularly preferred. It has been found that polyol contents of at least 10% by weight surprisingly lead to a finer particle size distribution and positively influence the stability of the emulsions.

Carbohydrates

The compounds optionally used as carbohydrates include all food-grade sugars such as, for example, glucose, sucrose, fructose, trehalose, maltose or lactose. Glucose and sucrose are preferably added to the emulsion. Carbohydrates may be present in the formulations in quantities of 0 to 70% by weight, preferably in quantities of 10 to 70% by weight and more particularly in quantities of 30 to 60% by weight, based on the emulsions.

As with the polyols, it has surprisingly been found that the use of carbohydrates in quantities of at least 10% by weight leads to smaller particle size distributions and improved stability.

Antioxidants

Tocopherol, tocopherol derivatives and/or ascorbic acid in quantities of 0 to 1% by weight, based on the total weight of the emulsion, may be added to the emulsions as antioxidants, i.e. to stabilize the emulsions against oxidation.

Aqueous Phase

The aqueous phase is normally formed by purified water which may optionally contain food-grade salts and other water-soluble additives. However, as already described under the production processes, fruit or vegetable juices, fruit juice beverages, milk, mixed milk beverages, sports beverages, may also be directly used as the aqueous phase. This phase is preferably adjusted to a pH below 7 and more particularly to a pH below 4 before emulsification. In fruit juices in particular, the adjustment to an acidic pH contributes to improved stability of the final formulation. The pH may be adjusted with typical food-grade acids, such as dilute hydrochloric acid or citric acid for example. The final emulsion should have a pH below 8 and preferably below 6.5.

Emulsion Formulations

The following emulsion formulations are preferred for their improved long-term stability and processability in beverages or dairy products:

A) Emulsions Containing

a) 0.5 to 40% by weight conjugated linoleic acid or derivatives of conjugated linoleic acid
b) 0.2 to 20% by weight vegetable lecithin mixtures
c) 0 to 20% by weight co-emulsifiers
d) 0 to 70% by weight polyols
e) 0 to 70% by weight carbohydrates
f) 0 to 1% by weight antioxidants
g) 20 to 99% by weight aqueous phase, with the proviso that the emulsions contain no other oils and fats except for the triglycerides of components a), b) and/or c) optionally present.
   B) Emulsions Containing
a) 0.5 to 40% by weight conjugated linoleic acid or derivatives of conjugated linoleic acid
b) 0.2 to 20% by weight vegetable lecithin mixtures
c) 0 to 20% by weight co-emulsifiers
d) 10 to 70% by weight polyols
e) 0 to 70% by weight carbohydrates
f) 0 to 1% by weight antioxidants
g) 20 to 89% by weight aqueous phase, with the proviso that the emulsions contain no other oils and fats except for the triglycerides of components a), b) and/or c) optionally present.
   C) Emulsions Containing
a) 0.5 to 40% by weight conjugated linoleic acid or derivatives of conjugated linoleic acid
b) 0.2 to 20% by weight vegetable lecithin mixtures
c) 0 to 20% by weight co-emulsifiers
d) 0 to 70% by weight polyols
e) 10 to 70% by weight carbohydrates
f) 0 to 1% by weight antioxidants
g) 20 to 89% by weight aqueous phase, with the proviso that the emulsions contain no other oils and fats except for the triglycerides of components a), b) and/or c) optionally present.
   D) Emulsions Containing
a) 0.5 to 40% by weight conjugated linoleic acid or derivatives of conjugated linoleic acid
b) 0.2 to 20% by weight vegetable lecithin mixtures
c) 0.1 to 15% by weight co-emulsifiers
d) 0 to 70% by weight polyols
e) 0 to 70% by weight carbohydrates
f) 0 to 1% by weight antioxidants
g) 20 to 99% by weight aqueous phase, with the proviso that the emulsions contain no other oils and fats except for the triglycerides of components a), b) and/or c) optionally present.
   E) Emulsions Containing
a) 0.5 to 40% by weight conjugated linoleic acid or derivatives of conjugated linoleic acid
b) 0.2 to 20% by weight vegetable lecithin mixtures
c) 0.1 to 15% by weight co-emulsifiers
d) 10 to 70% by weight polyols
e) 0 to 70% by weight carbohydrates
f) 0 to 1% by weight antioxidants
g) 20 to 89% by weight aqueous phase, with the proviso that the emulsions contain no other oils and fats except for the triglycerides of components a), b) and/or c) optionally present.
   F) Emulsions Containing
a) 0.5 to 40% by weight conjugated linoleic acid or derivatives of conjugated linoleic acid
b) 0.2 to 20% by weight vegetable lecithin mixtures
c) 0.1 to 15% by weight co-emulsifiers
d) 0 to 70% by weight polyols
e) 10 to 70% by weight carbohydrates
f) 0 to 1% by weight antioxidants
g) 20 to 89% by weight aqueous phase, with the proviso that the emulsions contain no other oils and fats except for the triglycerides of components a), b) and/or c) optionally present.

Emulsions with the following compositions are particularly preferred:

G) Emulsions Containing

a) 1 to 30% by weight conjugated linoleic acid or derivatives of conjugated linoleic acid
b) 0.5 to 10% by weight vegetable lecithin mixtures
c) 0 to 15% by weight co-emulsifiers
d) 0 to 70% by weight polyols
e) 0 to 70% by weight carbohydrates
f) 0 to 1% by weight antioxidants
g) 20 to 98% by weight aqueous phase, with the proviso that the emulsions contain no other oils and fats except for the triglycerides of components a), b) and/or c) optionally present.
   H) Emulsions Containing
a) 1 to 30% by weight conjugated linoleic acid or derivatives of conjugated linoleic acid
b) 0.5 to 10% by weight vegetable lecithin mixtures
c) 0 to 15% by weight co-emulsifiers
d) 10 to 70% by weight polyols
e) 0 to 70% by weight carbohydrates
f) 0 to 1% by weight antioxidants
g) 20 to 88% by weight aqueous phase, with the proviso that the emulsions contain no other oils and fats except for the triglycerides of components a), b) and/or c) optionally present.
   I) Emulsions Containing
a) 1 to 30% by weight conjugated linoleic acid or derivatives of conjugated linoleic acid
b) 0.5 to 10% by weight vegetable lecithin mixtures
c) 0 to 15% by weight co-emulsifiers
d) 0 to 70% by weight polyols
e) 10 to 70% by weight carbohydrates
f) 0 to 1% by weight antioxidants
g) 20 to 88% by weight aqueous phase, with the proviso that the emulsions contain no other oils and fats except for the triglycerides of components a), b) and/or c) optionally present.
   J) Emulsions Containing
a) 1 to 30% by weight conjugated linoleic acid or derivatives of conjugated linoleic acid
b) 0.5 to 10% by weight vegetable lecithin mixtures
c) 0.1 to 15% by weight co-emulsifiers
d) 0 to 70% by weight polyols
e) 0 to 70% by weight carbohydrates
f) 0 to 1% by weight antioxidants
g) 20 to 98% by weight aqueous phase, with the proviso that the emulsions contain no other oils and fats except for the triglycerides of components a), b) and/or c) optionally present.
   K) Emulsions Containing
a) 1 to 30% by weight conjugated linoleic acid or derivatives of conjugated linoleic acid
b) 0.5 to 10% by weight vegetable lecithin mixtures
c) 0.5 to 10% by weight co-emulsifiers
d) 10 to 70% by weight polyols
e) 0 to 70% by weight carbohydrates
f) 0 to 1% by weight antioxidants
g) 20 to 88% by weight aqueous phase, with the proviso that the emulsions contain no other oils and fats except for the triglycerides of components a), b) and/or c) optionally present.

Emulsions with the following compositions are especially preferred:

L) Emulsions Containing

a) 3 to 25% by weight conjugated linoleic acid or derivatives of conjugated linoleic acid
b) 0.5 to 10% by weight vegetable lecithin mixtures
c) 0 to 10% by weight co-emulsifiers
d) 0 to 70% by weight polyols
e) 0 to 70% by weight carbohydrates
f) 0 to 1% by weight antioxidants
g) 20 to 96% by weight aqueous phase, with the proviso that the emulsions contain no other oils and fats except for the triglycerides of components a), b) and/or c) optionally present.
   M) Emulsions Containing
a) 3 to 25% by weight conjugated linoleic acid or derivatives of conjugated linoleic acid
b) 0.5 to 10% by weight vegetable lecithin mixtures
c) 0.1 to 10% by weight co-emulsifiers
d) 0 to 70% by weight polyols
e) 0 to 70% by weight carbohydrates
f) 0 to 1% by weight antioxidants
g) 20 to 96% by weight aqueous phase, with the proviso that the emulsions contain no other oils and fats except for the triglycerides of components a), b) and/or c) optionally present.
   N) Emulsions Containing
a) 3 to 25% by weight conjugated linoleic acid or derivatives of conjugated linoleic acid
b) 0.5 to 10% by weight vegetable lecithin mixtures
c) 0.5 to 10% by weight co-emulsifiers
d) 30 to 60% by weight polyols
e) 0 to 70% by weight carbohydrates
f) 0 to 1% by weight antioxidants
g) 20 to 66% by weight aqueous phase, with the proviso that the emulsions contain no other oils and fats except for the triglycerides of components a), b) and/or c) optionally present.
   O) Emulsions Containing
a) 3 to 25% by weight conjugated linoleic acid or derivatives of conjugated linoleic acid
b) 0.5 to 10% by weight vegetable lecithin mixtures
c) 0.5 to 10% by weight co-emulsifiers
d) 0 to 70% by weight polyols
e) 30 to 60% by weight carbohydrates
f) 0 to 1% by weight antioxidants
g) 20 to 66% by weight aqueous phase, with the proviso that the emulsions contain no other oils and fats except for the triglycerides of components a), b) and/or c) optionally present.
   P) Emulsions Containing
a) 3 to 25% by weight triglycerides of conjugated linoleic acid
b) 0.5 to 10% by weight vegetable lecithin mixtures
c) 0 to 10% by weight co-emulsifiers
d) 30 to 60% by weight polyols
e) 0 to 70% by weight carbohydrates
f) 0 to 1% by weight antioxidants
g) 20 to 66% by weight aqueous phase, with the proviso that the emulsions contain no other oils and fats except for the triglycerides of components a), b) and/or c) optionally present.

### EXAMPLES

General Production (for Parameters, See Table 1)

Where it is included in the formulation, the co-emulsifier is dissolved or pre-dispersed in the aqueous phase as a whole with stirring at 50° C., optionally together with the polyols and/or the carbohydrates. The lecithin is then dissolved or dispersed in this warm (50° C.) phase with stirring. The CLA triglyceride or the free CLA is then added and pre-homogenized for 2 mins. at 5200 r.p.m.⁻¹ using an Ultra Turrax (IKA type 50; tool S50N-G40G). The preliminary emulsion is then homogenized in another homogenizing step using a high-pressure homogenizer (manufacturer: APV; LAB 1000; or LAB 60), generally at 750 bar (see Table 1). This process step is repeated in all up to 10 times (see Table 1).

Commercial Products Used:

| | |
|---|---|
| Premium ® IPM: | Lecithin from Cargill |
| Emultop ® HL 50: | Lecithin from Degussa |
| Lipoid ® S75: | Lecithin from Lipoid GmbH |
| Phospholipon ® 80: | Lecithin from Phospholipid GmbH |
| Lipotin ® NE: | Lecithin from Lucas Meyer |
| Sisterna ® SP 70: | Sucrose ester from Sisterna |
| Tonalin ® TG 80: | Triglyceride of conjugated linoleic acids from |
| | Cognis |
| Tonalin ® FFA 80: | free conjugated lionoleic acid from Cognis |
| Copherol ® 1250: | Tocopherolacetate from Cognis |

TABLE 1
Formulations and production of CLA-containing emulsions and beverages

| | | 1 | 2 | 3 | 4 | 5 | 6 |
|---|---|---|---|---|---|---|---|
| Water | [%] | 75.9 | 75.9 | 22.8 | 22.8 | 22.9 | 74.0 |
| Orange juice²⁾ | [%] | — | — | — | — | — | — |
| Orange juice concentrate 55° Brix | [%] | — | — | — | — | — | — |
| Tonalin ® FFA 80 | [%] | — | — | — | — | — | — |
| Tonalin ® TG 80 | [%] | 20.0 | 20.0 | 20.0 | 20.0 | 20.0 | 20.0 |
| Lecithin | | | | | | | |
| Premium ® IPM | [%] | — | — | — | — | — | — |
| Lipoid ® S75 | [%] | 4.0 | — | — | 4.0 | 4.0 | 4.0 |
| Phospholipon ® 80 | [%] | — | 4.0 | — | — | — | — |
| Emultop ® HL50 | [%] | — | — | 4.0 | — | — | — |
| Lipotin ® NE | [%] | — | — | — | — | — | — |
| Tween ® 60 | [%] | — | — | — | — | — | — |
| Tween ® 80 | [%] | — | — | — | — | — | 2.0 |
| Sisterna ® SP 70 | [%] | — | — | — | — | — | — |
| Copherol ® 1250 | [%] | 0.1 | 0.1 | 0.1 | 0.1 | — | — |
| Glycerol | [%] | — | — | 53.1 | 53.1 | — | — |
| Sucrose | [%] | — | — | — | — | 53.1 | — |
| Droplet size | | | | | | | |
| d₉₀ | [μm] | 0.232 | 0.227 | 0.281 | 0.127 | 0.134 | 0.186 |
| d_{3.2} | [μm] | 0.130 | 0.130 | 0.096 | 0.090 | 0.099 | 0.137 |
| High-pressure homogenizer | | LAB 1000 | LAB 1000 | LAB 1000 | LAB 1000 | LAB 1000 | LAB 1000 |
| Pressure | [bar] | 750 | 750 | 750 | 1000 | 1000 | 750 |
| No. of passes | | 5× | 5× | 5× | 5× | 10× | 5× |

| | | 7 | 8 | 9 | 10 | 11 | 12 |
|---|---|---|---|---|---|---|---|
| Water | [%] | 75.9 | 36.0 | 74.0¹⁾ | 32.9¹⁾ | 74.0 | — |
| Orange juice²⁾ | [%] | — | — | — | — | — | 70.0 |
| Orange juice concentrate 55° Brix | [%] | — | — | — | — | — | — |
| Tonalin ® FFA 80 | [%] | — | — | — | — | — | — |
| Tonalin ® TG 80 | [%] | 20.0 | 20.0 | 20.0 | 20.0 | 20.0 | 20.0 |
| Lecithin | | | | | | | |
| Premium ® IPM | [%] | — | — | — | — | 8.0 | — |
| Lipoid ® S75 | [%] | 4.0 | 4.0 | 4.0 | 4.0 | — | 4.0 |
| Phospholipon ® 80 | [%] | — | — | — | — | — | — |
| Emultop ® HL50 | [%] | — | — | — | — | — | — |
| Lipotin ® NE | [%] | — | — | — | — | — | — |
| Tween ® 60 | [%] | — | 2.0 | 2.0 | — | — | 2.0 |
| Tween ® 80 | [%] | — | — | — | — | — | — |
| Sisterna ® SP 70 | [%] | — | — | — | — | 2.0 | — |
| Copherol ® 1250 | [%] | 0.1 | — | — | — | — | — |
| Glycerol | [%] | — | 38.0 | — | — | — | — |
| Sucrose | [%] | — | — | — | — | 53.1 | — |
| Droplet size | | | | | | | |
| d₉₀ | [μm] | 0.135 | 0.200 | 0.117 | 0.238 | 0.208 | 0.216 |
| d_{3.2} | [μm] | 0.101 | 0.132 | 0.079 | 0.123 | 0.134 | 0.146 |
| High-pressure homogenizer | | LAB 1000 | LAB 1000 | LAB 60 | LAB 1000 | LAB 1000 | LAB 60 |
| Pressure | [bar] | 750 | 750 | 750/50 | 750 | 750 | 750/50 |
| No. of passes | | 5× | 5× | 10× | 5× | 5× | 10× |

| | | 13 | 14 | 15 | 16 | 17 | 18 | 19 |
|---|---|---|---|---|---|---|---|---|
| Water | [%] | 74.0 | 74.0 | 61.0 | 72.0 | 48.0 | 74.0 | — |
| Orange juice²⁾ | [%] | — | — | — | — | — | — | — |
| Orange juice concentrate 55° Brix | [%] | — | — | — | — | — | — | 74.0 |
| Tonalin ® FFA 80 | [%] | — | — | — | — | — | 20.0 | — |
| Tonalin ® TG 80 | [%] | 20.0 | 20.0 | 30.0 | 20.0 | 40.0 | — | 20.0 |
| Lecithin | | | | | | | | |
| Premium ® IPM | [%] | 4.0 | 4.0 | 6.0 | — | 8.0 | 4.0 | 4.0 |
| Lipoid ® S75 | [%] | | | — | — | — | — | — |
| | | | | | | | — | |
| Phospholipon ® 80 | [%] | — | — | — | — | — | — | — |
| | | | | | | | — | |
| Emultop ® HL50 | [%] | — | — | — | — | — | — | — |
| | | | | | | | — | |
| Lipotin ® NE | [%] | — | — | — | 6.0 | — | — | — |
| Tween ® 60 | [%] | — | 2.0 | 3.0 | 2.0 | 4.0 | 2.0 | 2.0 |
| Tween ® 80 | [%] | — | — | — | — | — | — | — |
| Sisterna ® SP 70 | [%] | 2.0 | — | — | — | — | — | — |
| Copherol ® 1250 | [%] | — | — | — | — | — | — | — |
| Glycerol | [%] | — | — | — | — | — | — | — |
| Droplet size | | | | | | | | |
| d₉₀ | [μm] | 0.234 | 0.196 | 0.159 | 0.227 | 5.827 | 0.185 | 2.799 |
| d_{3.2} | [μm] | 0.126 | 0.138 | 0.124 | 0.118 | 3.656 | 0.143 | 0.593 |
| High-pressure homogenizer | | LAB 60 | LAB 60 | LAB 60 | LAB 60 | — | LAB 60 | LAB 60 |
| Pressure | [bar] | 750/50 | 750/50 | 750/50 | 750/50 | — | 750/50 | 750/50 |
| No. of passes | | 10× | 10× | 10× | 1× | — | 10× | 2× |

Subsequent Characterization
Centrifuge Test

The freshly prepared emulsion was diluted with distilled water to a concentration of 1% by weight CLA and subjected to a centrifuge test for 5 mins. at 5,000 r.p.m. (Labofuge A centrifuge, manufacturer: Heraeus Sepatech). The physical stability of the emulsions was then evaluated by visual monitoring of the phase distribution (Table 2).

Particle Size Determination

The particle size distribution was measured with a Beckman Coulter LS 230 using the optical model emulsion.rfd PIDS inclded (of 14.08.01) in accordance with the operating instructions (1994). Water was used as the measuring medium. The particle size measurements were carried out immediately after production of the dispersions. Dilute emulsions with a concentration of 1% by weight CLA were prepared, i.e. a corresponding quantity of the concentrated CLA emulsions was stirred into distilled water (Table 1).

Storage Test

Selected dispersions were subjected to a storage test.

To investigate storage stability, the CLA emulsions—in the form of a 1% by weight solution in distilled water—were stored for 3 weeks under 3 different conditions and phase distribution was visually evaluated (Table 3).
1. Room temperature
2. Drying cabinet (ca. 40° C.)
3. Refrigerator (ca. 8° C.).

Results

TABLE 2
Centrifuge Test

| Emulsion | Water/Emulsion |
|---|---|
| 1 | Stable, no separation |
| 8 | Stable, no separation |
| 10 | Stable, no separation |
| 12 | Stable, no separation |
| 13 | Stable, no separation |
| 14 | Stable, no separation |

Storage Test

TABLE 3
Visual Evaluation of the Beverage Emulsions

| | Beginning of | | |
|---|---|---|---|
| Example | storage | After 1 week | After 3 weeks |
| 1 - Water - 8° C. | White, cloudy | Slight deposit, | Slight ring |
| | fine-particle | ring formation | formation, O.K. |
| 1 - Water - 20° C. | dispersion | Slight deposit, | Ring formation, |
| | | ring formation | O.K. |
| 1 - water - 40° C. | | Slight ring | Slight ring |
| | | formation, | formation, O.K. |
| | | sediment | |
| 4 - Water - 8° C. | White, cloudy | Slight ring | O.K. |
| | fine-particle | formation | |
| 4 - Water - 20° C. | dispersion | Slight ring | Ring formation, |
| | | formation | O.K. |
| 4 - Water - 40° C. | | Slight ring | Slight ring |
| | | formation | formation, O.K. |
| 5 - Water - 8° C. | White, cloudy | Slight ring | O.K. |
| | fine-particle | formation | |
| 5 - Water - 20° C. | dispersion | Slight separation, | Flocculation |
| | | ring formation, | |
| | | sediment | |
| 5 - Water - 40° C. | | Stable | Flocculation |
| 7 - Water - 8° C. | White, cloudy | Stable | O.K. |
| 7 - Water - 20° C. | fine-particle | Slight ring | Slight ring |
| | dispersion | formation | formation, O.K. |
| 7 - Water - 40° C. | | Slight ring | O.K. |
| | | formation | |
| 8 - Water - 8° C. | White, cloudy | Slight Ring | O.K. |
| | fine-particle | formation | |
| 8 - Water - 20° C. | dispersion | Slight ring | Ring formation, |
| | | formation | sediment |
| 8 - Water - 40° C. | | Slight ring | Ring formation, |
| | | formation | O.K. |
| 9 - Water - 8° C. | White, cloudy | Stable | O.K. |
| 9 - Water - 20° C. | fine-particle | Slight ring | Slight Ring |
| | dispersion | formation | formation, |
| | | | sediment |
| 9 - Water - 40° C. | | Slight ring | Ring formation, |
| | | formation | O.K. |

What is claimed is:

1. An emulsion comprising, based on the total weight of the emulsion,
a) 0.1 to 50% by weight of a conjugated linoleic acid or derivative thereof,
b) 0.1 to 30% by weight of at least one vegetable lecithin mixture,
c) 0 to 20% by weight of at least one co-emulsifier,
d) 0 to 70% by weight of at least one polyol,
e) 0 to 70% by weight of at least one carbohydrate,
f) 0 to 1% by weight of at least one antioxidant, and
g) 20 to 99% by weight of an aqueous phase, with the proviso that the emulsion contains no other oils or fats except for the triglycerides of components a), b) or c) if present.

2. The emulsion according to claim 1, wherein component a) is present in the amount of 0.5 to 30% by weight and component b) is present in the amount of 0.2 to 20% by weight.

3. The emulsion according to claim 1, wherein component a) comprises one or more triglycerides of one or more conjugated linoleic acids.

4. The emulsion according to claim 1, wherein the conjugated linoleic acid of component a) substantially comprises the C18:2 cis-9, trans-11 and C18:2 trans-10, cis-12 isomers of linoleic acid.

5. The emulsion according to claim 1, wherein the lecithins of component b) have a phosphatidyl choline content of 75% or less.

6. The emulsion according to claim 1, comprising at least one of a co-emulsifier including at least one polysorbate, a polyol selected from glycerol and sorbitol, and a carbohydrate selected from glucose and sucrose.

7. The emulsion according to claim 1, wherein the at least one co-emulsifier is present in the amount of 0.05 to 15% by weight.

8. The emulsion according to claim 1, comprising at least one of component d) and e) in the amount of at least 10% by weight.

9. The emulsion according to claim 1, having a d₉₀ particle size distribution of 0.117 to 0.281 μm, as measured with a Beckman Coulter LS 230 using the optical model emulsion.rfd PIDS included (of 14.08.01).

10. A beverage or dairy product containing an emulsion comprising, based on the total weight of the emulsion,
a) 0.1 to 50% by weight of a conjugated linoleic acid or derivative thereof,
b) 0.1 to 30% by weight of at least one vegetable lecithin mixture,
c) 0 to 20% by weight of at least one co-emulsifier,
d) 0 to 70% by weight of at least one polyol,
e) 0 to 70% by weight of at least one carbohydrate,
f) 0 to 1% by weight of at least one antioxidant, and
g) 20 to 99% by weight of an aqueous phase, with the proviso that the emulsion contains no other oils or fats except for the triglycerides of components a), b) or c) if present.

11. The beverage or dairy product according to claim 10, containing 0.1 to 20% by weight of the emulsion, based on the total weight of the beverage or dairy product.

12. The beverage or dairy product according to claim 10, wherein the emulsion comprises 0.5 to 40% of component a), 0.2 to 20% of component b), 0.1 to 15% of component c), and at least one of components d) and e) in the amount of at least 10%.

13. The beverage or dairy product according to claim 10, wherein component a) of the emulsion comprises one or more triglycerides of one or more conjugated linoleic acids.

14. The beverage or dairy product according to claim 10, wherein the conjugated linoleic acid of component a) substantially comprises the C18:2 cis-9, trans-11 and C18:2 trans-10, cis-12 isomers of linoleic acid.

15. The beverage or dairy product according to claim 10, wherein the lecithins of component b) have a phosphatidyl choline content of 75% or less.

16. The beverage or dairy product according to claim 10, wherein the emulsion comprises at least one of a co-emulsifier including at least one polysorbate, a polyol selected from glycerol and sorbitol, and a carbohydrate selected from glucose and sucrose.

17. The beverage or dairy product according to claim 10, wherein the emulsion has a d₉₀ particle size distribution of 0.117 to 0.281 μm, as measured with a Beckman Coulter LS 230 using the optical model emulsion.rfd PIDS included (of 14.08.01).

18. A process for the production of a beverage containing a conjugated linoleic acid or derivative thereof, comprising
a) directly pre-homogenizing an emulsion containing a conjugated linoleic acid or derivative thereof and an emulsifier in the beverage as an aqueous phase on the rotor/stator principle and
b) subsequently subjecting the resulting aqueous phase emulsion to high-pressure homogenization.

19. The process according to claim 18, wherein step b) is repeated to a total energy input of about 1×10⁵to 2×10⁸ J m−3.

20. The process according to claim 18, wherein the resulting emulsion has a d₉₀ particle size distribution of 0.117 to 0.281 μm, as measured with a Beckman Coulter LS 230 using the optical model emulsion.rfd PIDS included (of 14.08.01).

21. The process according to claim 18, wherein the emulsifier comprises a vegetable lecithin mixture and, optionally, a co-emulsifier.

22. The process according to claim 18, wherein the conjugated linoleic acid or derivative thereof comprises one or more triglycerides of one or more conjugated linoleic acids.
